(19) United States
(12) Patent Application Publication
(10) Pub. No.: US 2006/0135926 A1
(43) Pub. Date: Jun. 22, 2006

(54) Wrapper component for personal care articles having an easy opening and closing feature

(75) Inventors: Daniel William Dick, Manawa, WI (US); Sheila Marie Heyrman, Appleton, WI (US); Jennifer Lynn Marvin, Greenville, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/013,906

(22) Filed: Dec. 16, 2004

(51) Int. Cl. A61F 13/15

(52) U.S. Cl. 604/385.02

(57) ABSTRACT

The present invention provides a wrapped personal care product having a wrapper component and a personal care product placed within the wrapper component. The wrapper component has a chamber, an opening having a first side and a second side which reveals the chamber when opened but conceals the chamber when closed, and an opening device. The opening device has a first shape memory element on the first side of the opening and a second shape memory element on the second side of the opening. Each shape memory element has a first end and a second end and the first end of the first shape memory element is adjacent the first end of the second shape memory element and the second end of the first shape memory element is adjacent the second end of the second shape memory element. When in the relaxed state, each shape memory element has a first shape such that the opening is in a closed position and a second shape when subject to a compression force from the first end and second end of the each shape memory elements simultaneously such that the opening is opened revealing the chamber. Also disclosed is a method for disposing of used absorbent personal care articles.

### FIELD OF THE INVENTION

The present invention relates generally to a personal care product, and in particular, to a wrapper component for a personal care article that includes an easy opening and closing feature which aids the user in removing an unused personal care product from the wrapper component and inserting a used product into the wrapper component for disposal.

### BACKGROUND OF THE INVENTION

Absorbent articles such as pantiliners, feminine napkins and tampons are frequently carried about in purses, backpacks, briefcases, and the like until needed. Typically, the articles are placed in purses, backpacks, briefcases, and the like such that they are loose, and are free to move with in the purses, backpacks, briefcases, and the like with the other items contained therein. Unfortunately, purses, backpacks, briefcases, and the like do not always provide a hygienic environment for the personal care articles, and thus the personal care articles can become dirty and/or damaged. Further, the personal care articles can become scattered about in purses, backpacks, briefcases, and the like such that they are difficult to find when needed.

In the past, specifically designed pouches have been distributed for holding several personal care articles at a time. These pouches reduce contact between the personal care articles and potentially non-hygienic environments, and make the personal care articles easier to find when needed. The pouches are made from durable materials such as heavy vinyl so they can be reused, but reuse necessitates that the pouches be refilled from time to time. Further, the pouches frequently become non-hygienic after extended use, requiring them to be cleaned or discarded and replaced.

To avoid the problems described above, often personal care articles, including for example feminine pads, tampons and pantiliners, are individually wrapped in a wrapper component, pouch or similar package. Currently available wrapper components have one of more layers of a thin sheet or film of a thermoplastic material which is folded around the absorbent article and then sealed by the use of heat and/or pressure, ultrasonics, or an adhesive to form a package or a pouch with a flap. These wrapper components are designed to be open by breaking or tearing the material at or near a seal in order to subsequently remove the personal care articles from the wrappers. These current wrapper component configurations have many disadvantages. These disadvantages include: 1) the wrapper components are often destroyed when opened, making the wrapper component unsuitable for disposing of a used absorbent article; 2) the opened wrapper components do not adequately contain the used absorbent articles to prevent odors and fluids from being release when a used absorbent article is placed within the wrapper component after use, since the wrapper is often torn when opened; 3) it is often difficult to insert a used personal care article into the wrapper without soiling ones hands, since it is difficult to move any flaps out of the way or roll the used absorbent article in the piece of wrapper material; and/or 4) the current wrapper configuration can be difficult to open for older users and those user's with diseases which affect the dexterity of a user's hands, such as arthritis.

Another disadvantage of the current wrapper configuration is that the current commercially available wrappers do not provide privacy and discretion to the user of the absorbent articles. Current wrapper configurations are typically “loud” when opened since the wrapper material tends to make a “crinkling” and/or a “rustling” sound when the package is opened. This noise problem can be exasperated by a wrapper configuration where the wrapper also serves as a peel strip. As a result, when the packaging components for personal care articles are opened and initially used in public places, such as a public restroom, the current wrapper configurations tend to announce to others present in the restroom that the user is opening a personal care article, and that the user is in need of using of a personal care article.

Therefore, there is a need in the art for a wrapper component for a personal care article which is easy to open, is not destroyed when opened so that the wrapper component can be used to facilitate disposal of a used personal care article and provides discretion for the user during opening of the packaging component.

### BRIEF SUMMARY OF THE INVENTION

Generally stated, the present invention provides a wrapped personal care product having a wrapper component and a personal care product placed within the wrapper component. The wrapper component has a chamber, an opening having a first side and a second side which reveals the chamber when opened but conceals the chamber when closed, and an opening device. The opening device has a first shape memory element on the first side of the opening and a second shape memory element on the second side of the opening. Each shape memory element has a first end and a second end and the first end of the first shape memory element is adjacent the first end of the second shape memory element and the second end of the first shape memory element is adjacent the second end of the second shape memory element. When in the relaxed state, each shape memory element has a first shape such that the opening is in a closed position. When the first and second ends of each of the first and second shape memory elements are subjected to a compression force from the first end and second end of the each shape memory elements simultaneously, the opening is opened revealing the chamber. The compressive force causes each shape memory element to have a second shape which is different from shape of each shape memory device in the relaxed state.

In another embodiment of the present invention, the present invention provides for a method of disposing of a used personal care article. In this method, a used personal care product is placed within the chamber of a wrapper component with an opening having a first side and a second side which reveals a chamber, and an opening device. The opening device comprises a first shape memory element of a material on first side of the openings and a second shape memory element on the second side of the opening. Each shape memory element has a first end and a second end wherein the first end of the first shape memory element is adjacent the first end of the second shape memory element and the second end of the first shape memory element is adjacent the second end of the second shape memory element. Each shape memory element has a first shape while in the relaxed state such that the opening is in a closed position and a second shape when the shape memory elements are subjected to a compressive force from the first and second ends of the each shape memory element simultaneously such that the opening is opened revealing the chamber.

### BRIEF DESCRIPTION OF THE DRAWINGS

FIG. 1A shows a possible configuration for an absorbent personal care article placed in the wrapper component of the present invention.

FIG. 1B is a perspective view of a product component in a partially folded configuration.

FIG. 2 shows a perspective view of a wrapper component with a side seam opening in an open position.

FIG. 3 shows a partial cross-sectional view of the shape memory element placed within the wrapper material.

FIG. 4 shows a partial cross-sectional view of the shape memory element attached to the surface of the wrapper material.

FIG. 5 shows a wrapper cut away plan view of a wrapper component of the present invention in a closed position containing an absorbent personal care article.

FIG. 6A shows a perspective view of a wrapper component of the present invention with a top panel opening in an unopened state.

FIG. 6B shows a perspective view of a wrapper component of the present invention with a top panel opening in an opened state.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “personal care product” or “personal care article” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, personal care products, as used herein, includes without limitation diapers, child toilet training pants, adult incontinence garments, male incontinence products, tampons, vaginal suppositories, pantiliners, pads, sanitary napkins, interlabial pads, tissues, wipes, etc. For example, personal care products include without limitation Poise® feminine care products, including pantiliners and pads, and Kotex® feminine care products, including pads, tampons and liners, all available from Kimberly-Clark Corporation, Neenah, Wis.

As used herein, the term “nonwoven fabric or web” means a web having a structure of individual fibers or threads which are interlaid, but not in a regular or identifiable manner as in a knitted fabric. The term also includes individual filaments and strands, yarns or tows as well as foams and films that have been fibrillated, apertured, or otherwise treated to impart fabric-like properties. Nonwoven fabrics or webs have been formed from many processes such as for example, meltblowing processes, spunbonding processes, airlaying processes and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

### DETAILED DESCRIPTION OF THE INVENTION

Disposable absorbent articles such as, for example, feminine care and incontinent absorbent products, generally include a liquid pervious topsheet, a substantially liquid impervious backsheet, and an absorbent core positioned and held between the topsheet and the backsheet. The topsheet is generally operatively permeable to the liquids that are intended to be held or stored by the absorbent article, and the backsheet may be substantially impermeable or otherwise operatively impermeable to the liquids intended to be held or stored. In some configurations, such a pantiliners, the topsheet is an optional component. Disposable absorbent articles may also include other optional components or layers, such as liquid wicking layers, liquid distribution layers, barrier layers, and the like, as well as combinations thereof, which may improve the fluid handling and storage properties of the disposable absorbent article. Generally, disposable absorbent articles and the elements thereof provide a body-facing surface and a garment-facing surface, wherein the body-facing layer is the topsheet and the garment-facing layer is the backsheet. As an alternative, the substantially liquid impervious backsheet may be replaced with a liquid pervious backsheet, when the absorbent personal care article is used in conjunction with another liquid impervious layer or article, such as, for example liquid impervious pants.

In the present invention, the absorbent articles of the present invention are placed into a wrapper component with opening device which is easy to open and can be opened relatively quietly. Any absorbent article may be placed in the wrapper component, but typically, the wrapper component of the present invention will be used to hold sanitary napkins, tampons, pantiliners, incontinence pads and the like.

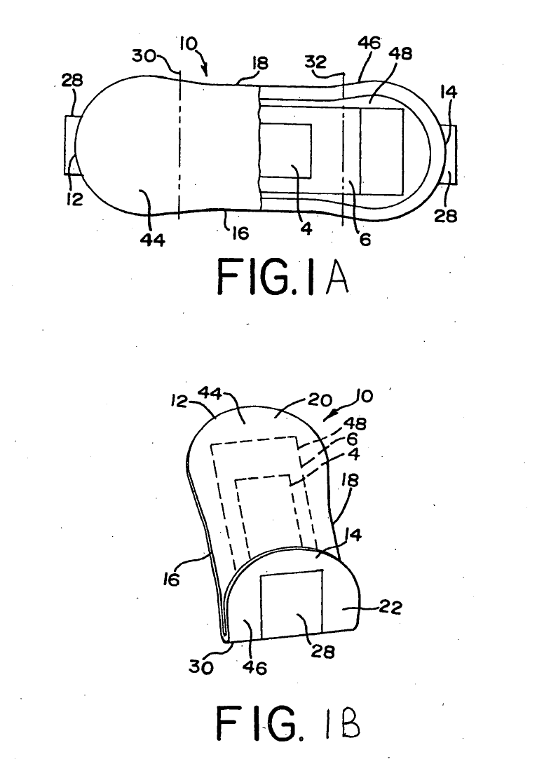

To obtain a better understanding of the absorbent articles which may be present in the packaging component of the present invention, attention is directed to FIGS. 1A and 1B. In FIGS. 1A and 1B, an exemplary absorbent product 10 is shown as including an outer cover 46 (otherwise referred to as a baffle or backsheet), an absorbent core 48, an optional tissue layer 6, an optional distribution layer 4 and a body side liner 44 (also referred to as the top sheet). The absorbent product 10 also has a first side 16 and a second side 18. The first and second sides 16, 18, respectively, are the longitudinal sides of the elongated absorbent product. The sides can be contoured, for example in a concave shape, or they can be linear. The sides can further include flaps (not shown) that extend laterally outward. Flaps are known in the art an are shown in, for example U.S. Pat. No. 6,387,084 issued to VanGompel et al, which is hereby incorporated by reference for its discussion of the flaps and flap attachment means and in its entirety. In one embodiment (not shown), one or more elastic elements are disposed along the sides to form a gasket with the body of the user. Elastic sides are known in the art, as is shown in U.S. Pat. No. 6,315,765 issued to Datta et al., which is hereby incorporated by reference for its discussion of the elastic sides and in its entirety. In one embodiment, the elastic elements are disposed between the liner and the outer cover.

The absorbent product 10 has a first body side surface 20 and a second garment side surface 22. Applied to at least a portion of the second garment side surface 22 is a garment attachment adhesive. In various embodiments, the garment attachment adhesive is configured as a single band of adhesive or as two or more spaced apart strips. Alternatively, the garment attachment adhesive includes a swirl pattern of adhesive which encompasses a major portion of the second garment surface 22 of the absorbent article 10.

A release strip 28, also known as a releasable peel strip, is removably secured to the garment attachment adhesive and serves to prevent premature contamination of the adhesive before the absorbent article 10 is secured to, for example, the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIGS. 1A and 1B.

The body side liner or topsheet 44, which is preferably liquid permeable, may be formed from one or more materials. The body side liner or topsheet 44 must be able to manage different body excretions depending on the type of product. In feminine care products, often the liner or body contacting layer must be able to handle menses and urine. In the present invention, the body side liner or topsheet 44 may include a layer constructed of any operative material, and may be a composite material. For example, the liner or body contacting layer can include a woven fabric, a nonwoven fabric, a polymeric film, a film-nonwoven fabric laminate or the like, as well as combinations thereof. Examples of a nonwoven fabric useable in the body side liner or topsheet 44 include, for example, an airlaid nonwoven web, spunbond nonwoven web, meltblown nonwoven web, a bonded-carded-web, hydroentangled nonwoven webs, spunlace webs or the like, as well as combinations thereof. Other examples of suitable materials for constructing the body side liner or topsheet 44 can include rayon, bonded carded webs of polyester, polypropylene, polyethylene, nylon, or other heat-bondable fibers finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat bondable materials.

Other examples of suitable materials for the body liner or topsheet 44 are composite materials of a polymeric film and a nonwoven fabric material. The composite materials are typically in the form of integral sheets generally formed by the extrusion of a polymer onto a nonwoven web, such as a spunbond material. In one arrangement, the liner or body contacting layer 44 can be configured to be operatively liquid-permeable with regard to the liquids that the article is intended to absorb or otherwise handle. The operative liquid-permeability may, for example, be provided by a plurality of pores, perforations, apertures or other openings, as well as combinations thereof, which are present or formed in the liner or body contacting layer. The apertures or other openings can help increase the rate at which bodily liquids can move through the thickness of the liner or body contacting layer and penetrate into the other components of the article (e.g. into the absorbent core 48). The selected arrangement of liquid-permeability is desirably present at least on an operative portion of the body side liner or topsheet 44 that is appointed for placement on the body-side of the article. The body side liner or topsheet 44 can provide comfort and conformability, and can function to direct bodily exudates away from the body and toward the absorbent core 48. The body side liner or topsheet 44 can be configured to retain little or no liquid in its structure, and can be configured to provide a relatively comfortable and non-irritating surface next to the body-tissues of a female wearer.

The baffle or backsheet 46 may include a layer constructed of any operative material, and may or may not have a selected level of liquid-permeability or liquid-impermeability, as desired. In a particular configuration, the baffle or backsheet 46 may be configured to provide an operatively liquid-impermeable baffle structure. The baffle or backsheet 46 may, for example, include a polymeric film, a woven fabric, a nonwoven fabric or the like, as well as combinations or composites thereof. For example, the baffle may include a polymeric film laminated to a woven or nonwoven fabric. In a particular feature, the polymeric film can be composed of polyethylene, polypropylene, polyester or the like, as well as combinations thereof. Additionally, the polymeric film may be micro-embossed, have a printed design, have a printed message to the consumer, and/or may be at least partially colored. Suitably, the baffle or backsheet 46 can operatively permit a sufficient passage of air and moisture vapor out of the article, particularly out of an absorbent (e.g. storage or absorbent core 48) while blocking the passage of bodily liquids. An example of a suitable baffle material can include a breathable, microporous film, such as those described in, for example U.S. Pat. No. 6,045,900 to McCormack et al.

Bicomponent films or other multi-component films can also be used, as well as woven and/or nonwoven fabrics which have been treated to render them operatively liquid-impermeable. Another suitable baffle material can include a closed cell polyolefin foam. Examples of closed cell foams include a closed cell polyethylene foam.

The liquid permeable body side liner 44 and the liquid-impermeable baffle 46 may be peripherally sealed together to enclose the absorbent core 48 to form the absorbent article 10. Alternatively, the body side liner or topsheet 44 can be wrapped around both the absorbent 48 and the baffle or backsheet 46 to form a wrapped pad. The body side liner 44 and baffle 46, and other components of the absorbent product, can be joined for example with adhesive bonds, sonic bonds, thermal bonds, pinning, stitching or any other attachment techniques known in the art, as well as combinations thereof.

The absorbent core 48 is designed to absorb bodily exudates, including menstrual fluid, blood, urine, and other body fluids. The absorbent core 48 may contain one or more layers of absorbent material. The layers can contain similar materials or different materials. Suitable materials for the absorbent core 48 include, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has good absorbency.

The absorbent core 48 can also be formed from a composite comprised of a hydrophilic material which may be formed from various natural or synthetic fibers, wood pulp fibers, regenerated cellulose or cotton fibers, or a blend of pulp and other fibers. In one embodiment of the present invention, the absorbent may be an airlaid material.

In one embodiment, the absorbent core 48 also includes a superabsorbent material, in addition to or in place of the hydrophilic material, which increases the ability of the absorbent core to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material may be biodegradable or bipolar. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may, for example, be by irradiation or covalent, ionic, Van der Waals, or hydrogen bonding. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Additional layers or substrates, including for example, the liquid acquisition and distribution layer 5, also referred to as a surge or transfer layer, and a tissue layer 6 are also incorporated into the absorbent product, for example between the body side liner or topsheet 44 and the absorbent core 48. The distribution layer 5 may be shorter than the absorbent core 48 or have the same length as the absorbent core 48. The distribution layer serves to temporarily hold an insulting fluid to allow the absorbent core sufficient time to absorb the fluid, especially when a superabsorbent material is present. In one embodiment, the absorbent core, transfer layer and other components, such as tissue layers, are free floating (unattached) between the outer cover and the liner, which are secured along only the peripheral edges thereof. Alternatively, the absorbent core, transfer layer and other components are attached to one or both of the outer cover and liner and/or to each other.

The absorbent article may be folded along a pair of fold lines 30, 32 to form a tri-fold configuration, prior to insertion into the wrapper component. In other embodiments, the absorbent article can be bi-folded, flat or rolled. In whichever configuration the absorbent article is in, the absorbent article is placed into a wrapper component of the present invention. Alternatively, a plurality (meaning two or more) of article components can be disposed in a single wrapper component and a plurality of wrapper components, with absorbent article contained therein, can be placed in an outer packaging component. The outer packaging component is any packaging which is used to transport, store, or protect the absorbent articles and wrapper components. Examples of outer packaging components include bags, boxes and the like. Outer packaging components are typically used to allow consumers a convenient means to transport a plurality absorbent articles from the store to their home or other places of use.

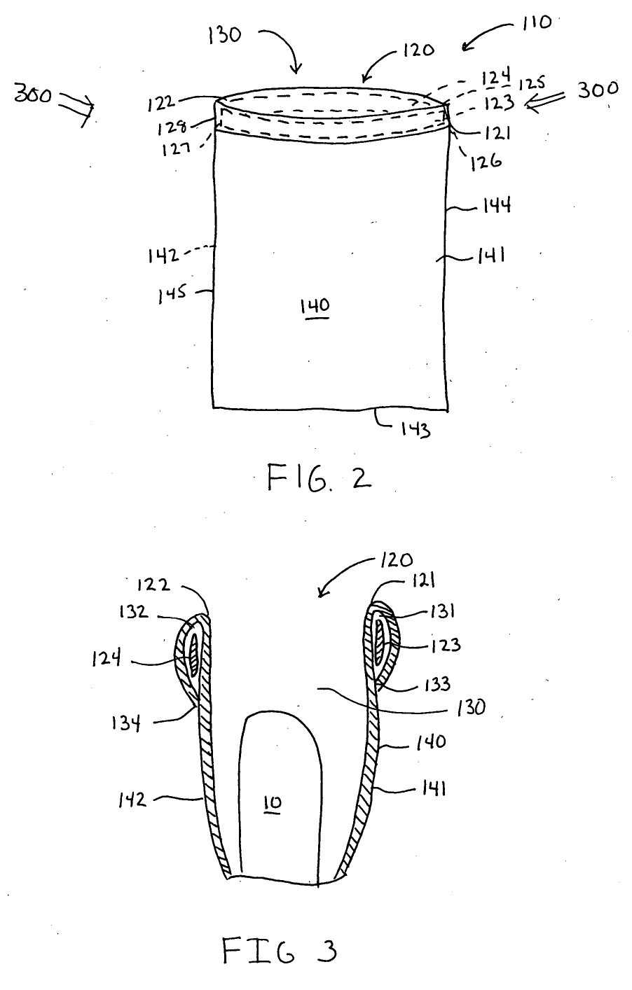

To gain a better understanding of the wrapper component of the present invention, attention is directed to FIGS. 2-5. FIG. 2 shows a perspective view of a wrapper component 110 having an opening 120 and being prepared from a material 140. The wrapper component 110 is shown in an open position and the opening 120, when opened, reveals a chamber 130, but conceals the chamber 130, when closed. This chamber 130 is adapted to hold at least one absorbent personal care article 10 (not shown in FIG. 2 but shown in FIG. 5). The opening 120, has a first side 121 and a second side 122. To aid in opening the opening 120, an opening device is provided. The opening device has a first shape memory element 123 on first side 121 of the opening and a second shape memory element 124 on the second side 122 of the opening. Each shape memory element 121, 122 has a first end 125, 126, respectively and a second end 127, 128, respectively. The first end 125 of the first shape memory element 121 is adjacent to the first end 126 of the second shape memory element 122 and the second end 127 of the first shape memory element 121 is adjacent the second end 128 of the second shape memory element 122. Each shape memory element 121, 122 has a first shape, when in a relaxed condition, such that the opening is in a closed position as shown in FIG. 5, thereby concealing the chamber 130. In additional, each shape memory device is capable of assuming a second shape, when subjected to a compressive force 300 from the first end 125, 126 to the second ends 127, 128 of the each shape memory element simultaneously, such that the opening 120 is in an open position, thereby revealing the chamber 130.

The chamber 130 is a defined space or cavity created between the material 140 used to form the wrapper component 110. In the simplest form, the wrapper component 140 has a first panel 141 and a second panel 142, as is shown in FIGS. 2, 3, 4 and 5. More than two panels may be used to form the wrapper component, which could result in a larger chamber being formed. If more than two panels are used, it is generally desired that the opening only has a first side 121 and a second side 122 for ease of opening the opening 120.

The first panel 141 and the second panel 142 each have a bottom edge 143, a first side edge 144, a second side edge 145 and a top edge 146. The bottom edge 143 of the first panel is adjoined to the bottom edge 143 of the second panel, the first side edge 144 of the first panel 141 is adjoined to the first side edge 141 of the second panel 142 and the second side edge 144 of the first panel 141 is adjoined to the second side edge 144 of the second panel 142. The opening 110 is located at the top edge 146 of the first panel 141 and the top edge 146 of the second panel 142 such that the first side 121 of the opening is located on the first panel 141 and the second side 122 of the opening is located on the second panel 142. Each panel 141 and 142 may be prepared from separate pieces of material or may be prepared from a unitary piece of material. In the case where the each panel 141 and 142 is made from separate pieces of material, the first and second side edges 143 and 144 of each material and bottom edge must be joined together, either directly or indirectly. An additional piece or pieces of material maybe present between each panel 141 and 142 where the panels are joined. Desirably, from the standpoint of cost, the first panel 141 and the second panel 142 are joined directly together. Any method known to those skilled in the art of joining two pieces of material together may be used. Exemplary joining methods include bonding, sealing, stitching and the like. Suitable bonding and sealing methods include, for example, but are not limited to adhesive bonding or sealing, bonding or sealing by the application of heat and pressure, ultrasonic bonding or sealing, or any other art known bonding methods. Alternatively, the material 140 used to prepare wrapper component 110 may be a unitary piece of material. In the case of a unitary piece of material, one of the side edges 144, 145 or the bottom edge 143 does not have to be bonded, sealed or otherwise joined to the corresponding edge on the other panel since the two panels are made from a single piece of material. In the present invention, it is desirable that the material 140 is prepared from a unitary piece of material such that the bottom edges 143 of the panels 141 and 142 do not have to be joined together. In alternative embodiment, the side edges 144, 145 and bottom edges 143 may be joined directly together or separated by one or more pieces of the same or different types of material. For example, the side edges could be joined together through an expandable material, such as an elastic nonwoven material to expand the side seams of the chamber 130. Other materials may be used just to increase the capacity of the chamber 130, or to aid in the bonding or sealing of the panels together.

The wrapper material 140 may be formed out from any pliable material such as a nonwoven materials, films, paper, coated paper, laminates, and/or cloth (including woven) materials, and combinations thereof. Suitable films include thermoplastic heat sealable films such as polyolefin films, e.g. polyethylene, polypropylene and blends thereof. Suitable nonwoven materials include spunbond nonwoven webs, meltblown nonwoven webs, and carded web nonwovens and nonwoven laminates. Suitable laminates useable in the present invention include, spunbond-spunbond laminate (SS), spunbond-meltblown-spunbond laminates (SMS), which are describe in U.S. Pat. No. 4,041,203, to Brock et al., and U.S. Pat. No. 5,464,688 to Timmons et al., both of which are hereby incorporated by reference. In addition, laminates of films and nonwovens and laminates of films may also be used as the material for the wrapper component. Examples of these other laminates include, for example, spunbond-film laminates (SF), and film-film laminates and other such laminates. In one embodiment, the material for the wrapper component made prepared from a film/spunbond laminate material available from Kimberly-Clark Corp, and know as HBSTL (“highly breathable stretch thermal laminate”), and which material is further disclosed in U.S. Pat. No. 6,276,032, the entire disclosure of which is hereby incorporated herein by reference. Other materials such as elastic films and elastic nonwoven webs may also be used to create a chamber which is expandable. Each panel maybe prepared from different materials and each panel maybe prepared form one or more material types. For example, one panel maybe a film and the other panel a nonwoven web. The wrapper material 140 may also be a multi-ply material or a single ply material.

The shape memory elements 123, 124 generally act as springs arm may be made from any stiff, elastically deformable material including plastic, metal, wood, paper, nonwovens, laminates of one or more of these materials and the like. The shape memory elements should have sufficient stiffness such that a compressive force needed to deform the shape memory elements is at least 5 grams (0.049 N) but usually less than about 750 grams (7.36 N). Generally, the force needed to deform the shape memory elements is in the range of about 25 grams (0.245N) to 500 grams (4.90N) and typically in the range of about 35 grams (0.343 N) to about 350 grams (3.43 N). The shape memory elements may be two separate elements or may be made from a single piece of material. If a single piece of material is used, element should lay flat and together in a relaxed state. Each shape memory element may be prepared from the same material or from different materials.

While in the relaxed state, with the opening 120 being in a closed position, the first and second shape memory elements 123, 124 each should have a generally linear contour and should lay flat together. In addition, the opening will have a generally linear contour, while in the closed position. When a compressive force 300 is applied to the shape memory elements to open the opening, each of the first and second shape memory elements 123, 124 assume a curved shape, such that the overall shape of the opening is oval in nature. Stated another way, the opening, when the shape memory elements are subjected to a compressive force 300 from the first end to the second end of each shape memory element, the first side of the opening and the second side of the opening are forced in opposing directions, creating a convex shape to the opening 120. When opened, the opening reveals the chamber 130 and an contents present in the chamber, in the case of FIG. 5, a personal care article 10.

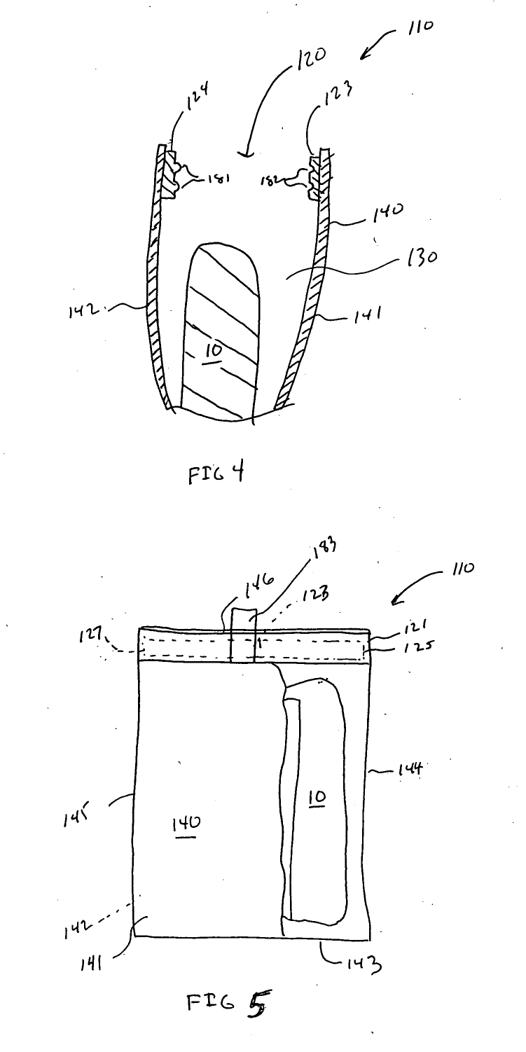

The shaped memory elements 123 and 124 may be located within the wrapper material 140, as is shown in FIG. 3 or may be directly or indirectly joined to the surface of the wrapper material 140, as is shown in FIG. 4. In FIG. 3, the shape memory elements 123 and 124 are placed between wrapper component material 140. In this configuration, the wrapper is formed to have pockets 131 and 132, on the first and second sides of the opening 121 and 122. The shape memory material is placed in each pocket and the pockets ends of the pockets are joined together, sealed or bonded in order to contain the shape memory elements 121 and 122 within the pockets 131 and 132. As such, the first shape memory element 121 is placed within the first side panel 141 proximate to said opening 120 and the second shape memory element 122 is placed within the second side panel 142 proximate to said opening 120. In this configuration, the shape memory elements 123 and 124 may be directly or indirectly attached to the wrapper material 140 or may be loose within the pockets. Method of attaching the shape memory elements include, for example, adhering with an adhesive means, such as adhesives or bonded to the wrapper material. In forming the configuration of the present invention shown in FIG. 3, the shape memory elements 123 and 124 may be placed within the pocket area 131 and 132 and the wrapper material 140 is folded over the shape memory element. Then a portion or all of the overlapping regions 133 and 134 of the wrapper material 140 is joined together, thereby containing the shape memory element within the pocket. Again the overlapping regions may be directly or indirectly joined together, using any of the joining methods described above for the side seams. In the alternative, the pockets may first be formed and the shape memory elements are slipped into an open end of the formed pocket, followed by joining, sealing or bonding the open end to contain the shape memory element within the pocket. The manner in which the shape memory elements 123 and 124 are placed and contained with the wrapper material 140 is not critical to the present invention.

In an alternative configuration, the first shape memory element 121 is attached directly or indirectly both the surface of the first side panel 141 proximate the opening 120 and the second shape memory element 122 is attached directly or indirectly to a surface of the second side panel 142 proximate to said opening, as is shown in FIG. 4. In this configuration, the shape memory elements may be placed on the surface of the wrapper material prior to joining the side edges and optionally the bottom edge of the wrapper component together, or the wrapper component may first be formed and the shape memory elements placed on the wrapper component after formation. From an ease of processing, the shape memory elements are desirably attached to the surface of the wrapper component 110 prior to joining the side edges 144, 145 and optionally the bottom edges 143 of the wrapper component 110 together using any of the above described joining means.

Another method of forming the shape memory elements does not require any additional material. In this aspect of the invention, the shape memory elements are integrally near the top portion of each side 141, 142 of the 140. The integral formed shape memory elements may be formed by folding at least a portion of the wrapper over itself at least one time or more times. The overlapped material may be joined to itself by using means such as an adhesive or bonding methods described above. As a result of the joining of the material together, the top portion of the material becomes stiffer, resulting in the top portion of the wrapper material becoming the shape memory element.

By placing the shape memory elements 123, 124 on the surface of the wrapper component 110 proximate the opening, the shape memory element may also provide a closure system to securely close the opening 120. The closure system provides a sealing means for the wrapper component 110. Suitable sealing means include tongue 181 and grove 182 sealing mechanisms, k-style sealing mechanisms, magnetic sealing mechanisms where the shape memory elements have magnetic properties. In the alternative, a sealing means may be provided in addition to the shape memory elements, whether the shape memory elements are placed within the wrapper material or are on the surface of the wrapper material. Other possible sealing means includes, for example, adhesive tape shown as 183 in FIG. 5, an adhesive applied to one or both sides of the opening, a snap, a button, a mechanical fastener (e.g., hook and loop), a tie, magnetic fasteners or any other device known by those skilled in the art. The fastening element can have various alternative shapes, including by not limited to a square, rectangle, triangle, circle, oval, oblong or diamond shape, or any other irregular shape or pattern. These separate sealing means can be placed adjacent the opening device, or above the opening device.

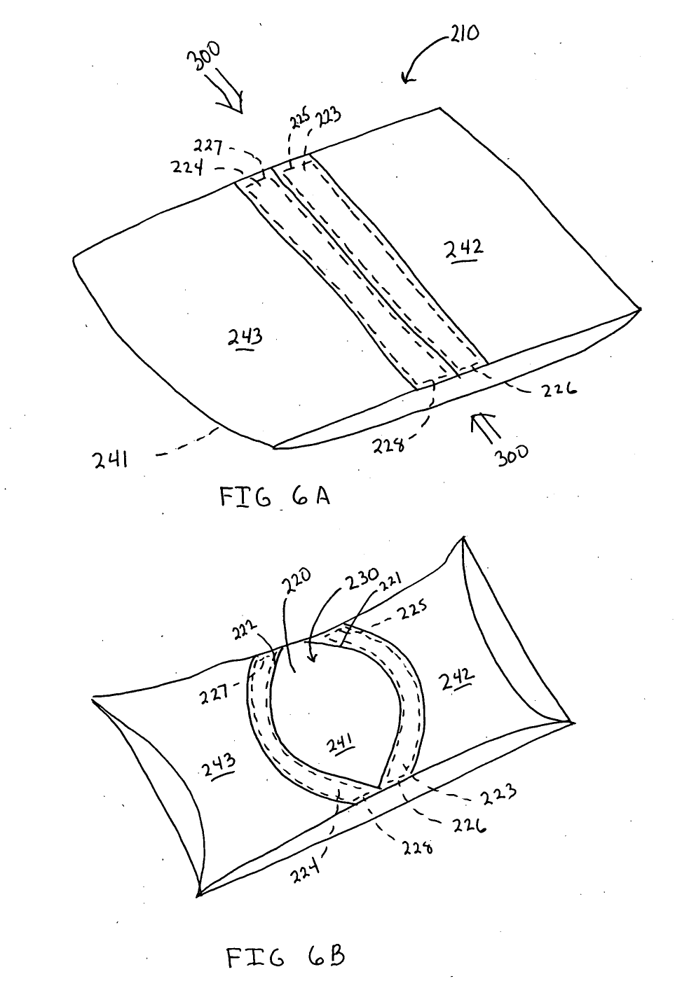

An alternative configuration for the wrapper component of the present invention is shown in FIGS. 6A and 6B. In this configuration, the wrapper component 210 has a bottom panel 241, a first top panel 242 and a second top panel 243. Each of the first top panel 242 and second top panel 243 is joined to the bottom panel 241 directly or indirectly such that the opening 220 is located between the first top panel 242 and the second top panel 243. A chamber 230 for holding an personal care article (not shown) is located between the top panels 242, 243 and the bottom panel 241. The opening 220 has a first side 221 and a second side 222 such that the first side of the opening 221 is located on the first top panel 242 and the second side of the opening 222 is located on the second top panel 243. Each of the top panels 242, 243 has a number of sides and all but the side of each top panel which is proximate the opening 220 is joined to, directly or indirectly to the back panel 241. Suitable joining methods include those described above and include adhesive, and bonding methods. Proximate the opening in each of the top panels is located a shape memory element. In this configuration, the first shape memory 223 is located on or in the first side panel 242 proximate the opening and second shape memory element 224 is located on or in the second side panel 243. The first shape memory element 223, has a first end 225 and a second end 226 and the second shape memory element 224, has a first end 227 and a second end 228. The first end 225 of the first shape memory element 223 is adjacent the first end 227 of the second shape memory element 224, and the second end 226 of the first shape memory element 223 is adjacent the second end 228 of the second shape memory element 224. In the relaxed state, the two shape memory elements generally have a linear contour and the opening 220 of the wrapper component 110 has a generally linear contour. In the configuration of FIG. 6A, when a compressive force 300 is applied to the opening device from the first ends 225, 227 of the first and second shape memory elements and from the second ends 226 and 228 of the first and second shape memory elements, the first and second shape memory elements assume a curved shape and tend to bow upwards and out from the plane in which the top panels 242 and 242 and away from the back panel to reveal the chamber 130, as is shown in FIG. 6B. When the opening 220 is opened, then the contents of the chamber 230 of the wrapper component 210 will be revealed, and in the case of the present invention reveal any personal articles present within the chamber 230. Similar to the wrapper component describe above and shown in FIG. 2, the shape memory elements may each be placed within pocket within the top side panels proximate to the opening, integrally formed in the top side panel material or attached directly or indirectly to the surface of each to the top side panels.

In the present invention, in order for the opening device to function properly, the ends of each shape memory elements should be adjacent to each other. By adjacent, it is meant that the first end of the first shape memory element is proximate the first end of the second shape memory element and the second ends of each memory element are likewise proximate each other. Desirably, the ends are located within the pockets or on the surface of the wrapper material such that when a user applies a compressive force to the opening device with their hand, the compressive force is applied to both shape memory elements at essentially the same time. To help ensure that the force is applied to both elements at the same time, it is desirable that the shape memory elements be approximately the same length. Further, it is desirable, but not required, that the shape memory elements be at least approximately the same width as the opening and more desirably, about the same overall width of the wrapper component. Optionally, the ends of the shape memory elements may be in contact with each other or joined together so that when a compressive force is applied to the shape memory elements the force is distributed to both elements and not just one shape memory element. As an alternative to joining or contacting the shape memory elements, when the shape memory elements are placed within pockets formed in the wrapper material, the edges of the pockets containing each shape memory elements on each side of the opening device may be joined together. In addition, the shape memory elements could be sealed or joined to the wrapper component along the edges sealed together.

The wrapper component of the present invention may used to hold a single absorbent personal care article, or may be used to hold more than one absorbent personal care articles. In addition, the wrapper component may be used to hold at least two different types of personal care article, for example, a pad and a liner, a pad and a tampon, or any other usable combination.

The size of the wrapper component should be larger than absorbent article and the opening, when in a compressed state, should be of a sufficient size for a user to easily remove the absorbent personal care product from the wrapper component. The actual size of the wrapper component and opening can easily be determined by those skilled in the art, and will depend on the type and size of the absorbent personal care article contained within the chamber. For example, a diaper would require a large wrapper component than a pantiliner. Generally, the width of the wrapper component should be from about 2 mm to about 50 mm wider that the overall width of the absorbent article, and the length of the wrapper component should be approximately the length of the absorbent personal care article plus the width of the opening device. Of course, if the absorbent personal care product is folded, the dimensions of the wrapper component should be adjusted accordingly. In addition, the length and width dimensions of the wrapper component may be adjusted with the width and length dimensions of the absorbent personal care article, if the absorbent personal care articles are placed in the wrapper component in a traverse direction.

To open the wrapper component of the present invention, a compression force is applied from the first end and the second end of each shape memory elements. When this compressive force is applied to the shape memory elements, each element assumes a curved shape or bowed shape, as is shown in FIGS. 2 and 6B. As a result of the bowed shape, the chamber of the wrapper component is revealed. When the compressive force is released, the shape memory elements again assume the relaxed position, which is essentially flat, thereby effectively closing the wrapper component. When the opening is in an opened state, the contents of the wrapper may be removed, or if the wrapper component is empty, a used absorbent personal care product may be inserted for disposal.

The present invention also provides a method of disposing of a used personal care article. In this method, a used personal care product in placed in a wrapper component provided with an unused personal care product. The wrapper component is described above. The shape memory elements are subject to a compression force from the first and second ends of the each shape memory elements simultaneously such that the opening is opened revealing the chamber. Once opened, an unused absorbent personal care product, if present, is removed, and then the used personal care product, desirably folded to a size which will fit into the chamber of the wrapper component, is inserted into the chamber and the compressive force applied to the shape memory elements is removed. When the compressive force is removed, the opening of the wrapper component is effectively closed. The used personal care product and the wrapper component may be disposed of in an appropriate manner.

As an optional feature, the wrapper component of the present invention may contain means for controlling odor from used absorbent personal care articles. Odor controlling means include odor masking, odor absorbing and odor containing. Examples of odor masking means include the addition of scents to the wrapper material either on an outside surface or on an interior surface. Odor absorbing means may include materials known to absorb odors such as activated charcoal and zeolites which can be placed within the raw materials used to prepare the wrapper material, so that the odor absorbing means are placed within the wrapper material, or the odor absorbing means maybe deposited in a coating applied to or into the wrapper material or place within a porous sack placed in the chamber of the wrapper component. Another way of controlling the odor is to prepare the wrapper component form a material which is not porous, or breathable, such as a film material.

As described above, the present invention provides a wrapper component for a personal care product which is easy to open to access the personal care product therein. In addition, the wrapper component is not destroyed when opened, leaving the wrapper component usable to facilitate disposal of a used personal care article. The wrapper component of the present invention also provides discretion for the user during opening of the packaging component by providing a relatively quiet opening mechanism.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A wrapped personal care product comprising:
a wrapper component comprising a chamber, an opening having a first side and a second side which reveals the chamber when opened but conceals the chamber when closed, and an opening device, wherein the opening device comprises a first shape memory element on first side of the opening and a second shape memory element on the second side of the opening, each shape memory element having a first end and a second end and the first end of the first shape memory element is adjacent the first end of the second shape memory element and the second end of the first shape memory element is adjacent the second end of the second shape memory element, each shape memory element having a first shape while in a relaxed state such that the opening is in a closed position in the relaxed state and a second shape when subject to a compression force from the first and second ends of the each piece of elements simultaneously such that the opening is opened revealing the chamber; and
a personal care product component disposed within the chamber of the wrapper.

2. The wrapped personal care product of claim 1, further comprising a first panel and a second panel, wherein the first and second panels are adjoined to form the chamber.

3. The wrapped personal care article of claim 2, wherein the first panel and the second panel each comprise a bottom edge, a first side edge, a second side edge and a top edge, wherein the bottom edge of the first panel is connected to the bottom edge of the second panel, the first side edge of the first panel is connected to the first side edge of the second panel and the second side edge of the first panel is connected to the second side edge of the second panel; and the opening is located at the top edge of the first panel and the top edge of the second panel such that the first side of the opening is located on the first panel and the second side of the opening is located on the second panel.

4. The wrapped personal care product of claim 3, wherein the first and second shape memory element each has a generally linear shape in a relaxed position and the opening of the wrapper has a generally linear shape in the relaxed position; and the first and second shape memory elements assume a curved shape when subject to a compressive force from the first end to the second end of each shape memory element such that the first side of the opening and the second side of the opening are forced in opposing directions to reveal the chamber of the wrapper component.

5. The wrapped personal care product of claim 4, wherein the first shape memory element is attached to a surface of the first side panel proximate said opening and the second shape memory element is attached a surface of the second side panel proximate to said opening.

6. The wrapped personal care product of claim 4, wherein the first shape memory element is placed within the first side panel proximate to said opening and the second shape memory element is placed within the second side panel proximate to said opening.

7. The wrapped personal care product of claim 1, further comprising a bottom panel, a first top panel and a second top panel, wherein each of the first top panel and second top panel is adjoined to the bottom panel such that the opening is located between the first top panel and the second top panel; the chamber is located between the top panels and the bottom panel; and the first side of the opening is located in the first top panel and the second side of the opening is located in the second top panel.

8. The wrapped personal care product of claim 7, wherein the first and second shape memory element each has a generally linear shape in a relaxed position and the opening of the wrapper has a generally linear shape in the relaxed position; and the first and second shape memory elements assume a curved shape when subject to a compressive force from the first end to the second end of each shape memory element such that the first side of the opening and the second side of the opening are forced in opposing directions to reveal the chamber of the wrapper component.

9. The wrapped personal care product of claim 8, wherein the first shape memory element is attached to a surface of the first side panel proximate said opening and the second shape memory element is attached a surface of the second side panel proximate to said opening.

10. The wrapped personal care product of claim 8, wherein the first shape memory element is placed within the first side panel proximate to said opening and the second shape memory element is placed within the second side panel proximate to said opening.

11. The wrapped personal care product of claim 1, wherein the wrapper component further comprises a sealing means.

12. The wrapped personal care product of claim 11, wherein the sealing means comprises a tongue and groove sealing mechanism.

13. The wrapped personal care product of claim 1, wherein the first and second shape memory elements also provide a means for sealing the wrapper.

14. The wrapped personal care product of claim 13, wherein the sealing means comprises a tongue and groove sealing mechanism.

15. The wrapped personal care product of claim 1, wherein the wrapper further comprises an odor adsorbing means or odor masking means.

16. The wrapped personal care product of claim 2, wherein the shape memory element is integral with said first and second panels.

17. The wrapped personal care product of claim 1, wherein the shape memory element comprises elastically deformable material comprising plastic, metal, wood, paper, nonwovens, or laminates of one or more of these materials.

18. The wrapped personal care product of claim 1, wherein said compression force to open the opening is between about 5 grams and about 750 grams.

19. The wrapped personal care product of claim 18, wherein said compression force to open the opening is between about 25 grams and about 500 grams.

20. A method of disposing of a used personal care article, comprising providing a wrapper component comprising, a chamber, an opening having a first side and a second side which reveals the chamber, and an opening device, wherein the opening device comprises a first shape memory element on first side of the openings and a second shape memory element on the second side of the opening, each shape memory element having a first end and a second end and the first end of the first shape memory element is adjacent the first end of the second shape memory element and the second end of the first shape memory element is adjacent the second end of the second shape memory element, each shape memory element comprising a first shape while in the relaxed state such that the opening is in a closed position and a second shape when subjected to a compressive force from the first end to the second end of the each strip simultaneously, such that the opening is opened revealing the chamber.
